SEQUENCE LISTING (29 sequences)

SEQ ID NO: 1 - DNA, 16 bp, synthetic construct
  source 1..16: mol_type = other DNA
tcataagacgaacgga

SEQ ID NO: 2 - DNA, 11 bp, synthetic construct
  source 1..11: mol_type = other DNA
aagctctcttc

SEQ ID NO: 3 - DNA, 30 bp, synthetic construct
  source 1..30: mol_type = other DNA
aagctctcttctgggggctgttggctctgg

SEQ ID NO: 4 - DNA, 30 bp, synthetic construct
  source 1..30: mol_type = other DNA
ctcttctgggggctgttggctctggtctgc

SEQ ID NO: 5 - DNA, 30 bp, synthetic construct
  source 1..30: mol_type = other DNA
ctgggggctgttggctctggtctgctctga

SEQ ID NO: 6 - DNA, 30 bp, synthetic construct
  source 1..30: mol_type = other DNA
ggctgttggctctggtctgctctgaagaaa

SEQ ID NO: 7 - DNA, 30 bp, synthetic construct
  source 1..30: mol_type = other DNA
ttggctctggtctgctctgaagaaaattcc

SEQ ID NO: 8 - DNA, 30 bp, synthetic construct
  source 1..30: mol_type = other DNA
tctggtctgctctgaagaaaattccctggc

SEQ ID NO: 9 - DNA, 30 bp, synthetic construct
  source 1..30: mol_type = other DNA
tctgctctgaagaaaattccctggccttcc

SEQ ID NO: 10 - DNA, 30 bp, synthetic construct
  source 1..30: mol_type = other DNA
tctgaagaaaattccctggccttcccttgt

SEQ ID NO: 11 - DNA, 30 bp, synthetic construct
  source 1..30: mol_type = other DNA
agaaaattccctggccttcccttgtaggaa

SEQ ID NO: 12 - DNA, 30 bp, synthetic construct
  source 1..30: mol_type = other DNA
attccctggccttcccttgtaggaagttct

SEQ ID NO: 13 - DNA, 30 bp, synthetic construct
  source 1..30: mol_type = other DNA
ctggccttcccttgtaggaagttctggggg

SEQ ID NO: 14 - DNA, 30 bp, synthetic construct
  source 1..30: mol_type = other DNA
cttcccttgtaggaagttctgggggctgtt

SEQ ID NO: 15 - DNA, 30 bp, synthetic construct
  source 1..30: mol_type = other DNA
cttgtaggaagttctgggggctgttggctc

SEQ ID NO: 16 - DNA, 30 bp, synthetic construct
  source 1..30: mol_type = other DNA
aggaagttctgggggctgttggctctggtc

SEQ ID NO: 17 - DNA, 30 bp, synthetic construct
  source 1..30: mol_type = other DNA
gttctgggggctgttggctctggtctgctc

SEQ ID NO: 18 - DNA, 30 bp, synthetic construct
  source 1..30: mol_type = other DNA
gggggctgttggctctggtctgctctgaag

SEQ ID NO: 19 - DNA, 30 bp, synthetic construct
  source 1..30: mol_type = other DNA
ctgttggctctggtctgctctgaagaaaat

SEQ ID NO: 20 - DNA, 29 bp, synthetic construct
  source 1..29: mol_type = other DNA
ggctctggtctgctctgaagaaaatccct

SEQ ID NO: 21 - DNA, 25 bp, synthetic construct
  source 1..25: mol_type = other DNA
tggtctgctctgaagaaaatccctg

SEQ ID NO: 22 - DNA, 116 bp, synthetic construct
  source 1..116: mol_type = other DNA
aagctctcttctgggggctgttggctctggtctgctctgaagaaaattccctggccttcccttgtaggaagttctgggggctgttggctctggtctgctctgaagaaaattccctg

SEQ ID NO: 23 - DNA, 60 bp, synthetic construct
  source 1..60: mol_type = other DNA
aagctctcttctgggggctgttggctctggtctgctctgaagaaaattccctggccttcc

SEQ ID NO: 24 - DNA, 61 bp, synthetic construct
  source 1..61: mol_type = other DNA
cttcccttgtaggaagttctgggggctgttggctctggtctgctctgaagaaaattccctg

SEQ ID NO: 25 - DNA, 116 bp, synthetic construct; identical to SEQ ID NO: 22
  source 1..116: mol_type = other DNA

SEQ ID NO: 26 - DNA, 19 bp, synthetic construct
  source 1..19: mol_type = other DNA
aagctctcttctgggggct

SEQ ID NO: 27 - DNA, 65 bp, synthetic construct
  source 1..65: mol_type = other DNA
gggctgttggctctggtctgctctgaagaaaattccctggccttcccttgtaggaagttctgggg

SEQ ID NO: 28 - DNA, 42 bp, synthetic construct
  source 1..42: mol_type = other DNA
tgggggctgttggctctggtctgctctgaagaaaattccctg

SEQ ID NO: 29 - DNA, 116 bp, synthetic construct; identical to SEQ ID NO: 22
  source 1..116: mol_type = other DNA

(19) United States
(12) Patent Application Publication
(10) Pub. No.: US 2025/0231172 A1
(43) Pub. Date: Jul. 17, 2025

(54) SINGLE MOLECULE MULTI-MOLECULAR TRACE METHODS AND SYSTEMS

(71) Applicant: Roche Sequencing Solutions, Inc., Pleasanton, CA (US)

(72) Inventors: Geoffrey Barrall, San Jose, CA (US); Eric Takeshi Harada, San Jose, CA (US); Jason David Komadina, Livermore, CA (US); John Mannion, Palo Alto, CA (US)

(21) Appl. No.: 19/097,390

(22) Filed: Apr. 1, 2025

Related U.S. Application Data
(63) Continuation of application No. PCT/EP2023/077003, filed on Sep. 29, 2023.
(60) Provisional application No. 63/412,774, filed on Oct. 3, 2022.

(51) Int. Cl. G01N 33/487; C12Q 1/6869

(52) U.S. Cl. CPC G01N 33/48721; C12Q 1/6869

(57) ABSTRACT

Methods of sequencing by expansion and related improvements to the sequencing of surrogate polymers in a nanopore are described. The surrogate polymer is formed from a template nucleic acid molecule. A surrogate polymer includes multiple units. Each unit includes a reporter code portion. The reporter codes correspond to the different nucleotides. surrogate polymers may get stuck in the nanopore. Embodiments described herein address these stuck surrogate polymers. In order to allow for multiple reads on the surrogate polymer, a processive consensus technique can be applied. The surrogate polymer may be moved a few units forward and then fewer units backward so that some of the same reporter codes are identified again. This method allows for multiple reads of the same reporter codes. The surrogate polymer eventually passes through the nanopore in the forward direction. Periodically, higher clearing voltages may be applied to clear any stuck surrogate polymer in the nanopore.

### CROSS-REFERENCE TO RELATED PATENT APPLICATIONS

This application is a continuation of International Patent Application No. PCT/EP2023/077003, filed Sep. 29, 2023, which claims the benefit of U.S. Provisional Patent Application No. 63/412,774, filed Oct. 3, 2022, which are hereby incorporated by reference in their entirety.

### STATEMENT REGARDING SEQUENCE LISTING

The Sequence Listing associated with this application is provided in xml format in lieu of a paper copy, and is hereby incorporated by reference into the specification. The name of the xml file containing the Sequence Listing is P37013 seq list.xml. The xml file is 27,648 bytes, was created on Oct. 31, 2023, and is being submitted electronically via Patent Center.

### TECHNICAL FIELD

Embodiments described herein relate to nanopore-based sequencing methods and systems. In particular, methods and systems include methods to generate consensus reads using Sequencing By eXpansion (SBX™).

### BACKGROUND

Nanopore membrane devices having pore sizes on the order of one nanometer in internal diameter have shown promise in rapid nucleotide sequencing. When a voltage potential is applied across a nanopore immersed in a conducting fluid, a small ion current attributed to the conduction of ions across the nanopore can exist. The size of the current is sensitive to the pore size and which molecule in the nanopore. The molecule can be a particular reporter code corresponding to a particular nucleotide, thereby allowing detection of a nucleotide at a particular position of a nucleic acid. A voltage or other signal in a circuit including the nanopore can be measured (e.g., at an integrating capacitor) as a way of measuring the resistance of the molecule, thereby allowing detection of which molecule is in the nanopore.

A nanopore based sequencing chip may be used for DNA sequencing. A nanopore based sequencing chip can incorporate a large number of sensor cells configured as an array. For example, an array of one million cells may include 1000 rows by 1000 columns of cells.

The signals that are measured can vary from chip to chip and from cell to cell of a same chip due to manufacturing variability. Therefore, it can be difficult to determine the correct molecule, which may be or correspond to the correct nucleotide in a particular nucleic acid or other polymer in a cell. In addition, other time dependent non-idealities in the measured signals can lead to inaccuracies. And, because these circuits employ biochemical circuit elements, e.g., lipid bilayers, nanopores, etc., the variability in the electrical characteristics can be much higher than for traditional semiconductor circuits. Further, sequencing processes are stochastic in nature, and thus variability can occur across a wide variety of systems, including sequencing devices not using nanopores.

Accordingly, improved characterization techniques are desired to improve the accuracy and stability of sequencing processes.

### BRIEF SUMMARY

Embodiments described herein involve methods of sequencing and improvements to the sequencing of surrogate polymers encoded with nucleic acid information in a nanopore. The surrogate polymer (also referred to herein as an “Xpandomer” polymer) is formed by template directed synthesis that preserves the original genetic information of the target nucleic acid, while also increasing linear separation of the individual elements of the sequence data. The surrogate polymer is formed from a template nucleic acid molecule. A surrogate polymer includes multiple units. Each unit includes a reporter code portion or portions. The reporter codes correspond to the different nucleotides (e.g., A, T, C, G). The reporter codes generate different electrical signals in the nanopore and therefore allow identification of the nucleotide sequence. Each unit includes a translocation control element (TCE). To pass through the nanopore, the TCE requires a higher voltage to be applied as compared to the baseline voltage for driving the rest of the unit through the nanopore. Surrogate polymers can be passed forward and backward through a nanopore several times to allow for multiple reads. Surrogate polymers include one leader segment, which may get stuck in a membrane on one side of the nanopore when backing the surrogate polymers out of the nanopore. Embodiments described herein address these stuck surrogate polymers.

In order to allow for multiple reads on the surrogate polymers, a technique of processive consensus can be applied. The surrogate polymer may be moved a few units forward (e.g., 30) and then fewer units backward (e.g., 25) so that some of the same reporter codes are identified again. This method allows for multiple reads of the same reporter codes. The surrogate polymereventually passes through the nanopore in the forward direction. Periodically, higher clearing voltages may be applied to clear any stuck surrogate polymer in the nanopore.

Clearing voltages may be applied more frequently but in a targeted manner. Cells (i.e., wells) where the surrogate polymer is not determined to be stuck within the nanopore may be deactivated before the clearing voltage is applied. Techniques allow molecules of any length can be sequenced at their full length with a depth of greater than one.

In view of the foregoing, an aspect of the present disclosure is a method for sequencing a target nucleic acid molecule, the method comprising applying a first number of voltage pulses at a first level across a nanopore to displace a compound a first distance in a first direction through the nanopore, the compound created from the target nucleic acid molecule, wherein the compound comprises a plurality of units, each unit of the plurality of units comprises one type of reporter element of a plurality of types of reporter elements, each type of reporter element corresponds to an identity of a nucleotide in the target nucleic acid molecule, and applying the first number of voltage pulses passes a first subset of the plurality of units through the nanopore; detecting, in the nanopore, the types of reporter elements in the first subset; applying a second number of voltage pulses at a second level across the nanopore to displace the compound a second distance in a second direction through the nanopore, wherein the first direction is opposite the second direction, the voltage pulses of the first number of voltage pulses have an opposite polarity as the voltage pulses of the second number of voltage pulses, the second distance is less than the first distance, and the second number is less than the first number; applying a third number of voltage pulses at a third level across the nanopore to displace the compound a third distance in the first direction through the nanopore, wherein applying the third number of voltage pulses passes a second subset of the plurality of units through the nanopore the second subset and the first subset comprise some of the same units, the second subset comprises units not in the first subset, and the third distance is greater than the second distance; and detecting, in the nanopore, the types of reporter elements in the second subset.

In some embodiments, the method further comprises applying a clearing voltage at a fourth level across the nanopore to pass the compound entirely out of the nanopore, wherein the fourth level is greater than the first level, the second level, and the third level.

In some embodiments the compound is a first compound of a plurality of compounds, the plurality of compounds is created from a plurality of target nucleic acid molecules, the nanopore is a first nanopore of a plurality of nanopores, and each compound of the plurality of compounds is in one nanopore of the plurality of nanopores, the method further comprising applying the first number of voltage pulses at the first level, the second number of voltage pulses at the second level, and the third number of voltage pulses at the third level to the plurality of nanopores.

In some embodiments, the method further comprises determining a plurality of sequences of the plurality of target nucleic acid molecules.

In some embodiments, the size distribution of the plurality of sequences has a mode greater than 300 nt.

In some embodiments, the method further comprises applying the first number of voltage pulses at the first level, the second number of voltage pulses at the second level, and the third number of voltage pulses at the third level to the plurality of nanopores; determining that a first portion of the plurality of compounds is being displaced in a first portion of the plurality of nanopores by the first number of voltage pulses, the second number of voltage pulses, or the third number of voltage pulses, applying a clearing voltage at a fourth level across each nanopore of a second portion of the plurality of nanopores to pass a second portion of the plurality of compounds entirely out of the respective nanopore of the plurality of nanopores, wherein the fourth level is greater than the first level, the second level, and the third level, and the second portion of the plurality of nanopores does not comprise nanopores in the first portion of the plurality of nanopores.

In some embodiments, the method further comprises determining a sequence of the target nucleic acid molecule.

In some embodiments, determining the sequence of the target nucleic acid molecule comprises for one or more units in both the first subset and the second subset, detecting the same type of reporter element.

In some embodiments, the method further comprises passing the compound entirely out of the nanopore.

In some embodiments, passing the compound entirely out of the nanopore occurs during the applying of the third number of voltage pulses.

In some embodiments, each unit of the plurality of units comprises a translocation control element, applying the first number of voltage pulses passes a first number of translocation control elements through the nanopore, and the first number of voltage pulses equals the first number of translocation control elements.

In some embodiments, the method further comprises applying a voltage at a fourth level across the nanopore to displace the compound a fourth distance in the first direction through the nanopore in between voltage pulses of the first number of voltage pulses, wherein the voltage at the fourth level is the same polarity as the voltage pulses of the first number of voltage pulses, the fourth level is less than the first level, and the compound after being displaced the fourth distance has a translocation control element in the nanopore.

In some embodiments, the second level is greater than the first level.

In some embodiments, the method further comprises measuring signal values for a nanopore having a voltage applied across the nanopore when reporter elements in the first subset of the plurality of units are in the nanopore; and determining, using the signal values, the types of reporter elements in the first subset, thereby determining the identities of nucleotides in the target nucleic acid molecule.

In some embodiments, the first subset of the plurality of units comprises 30 or more units.

In some embodiments, the third level is equal to the first level.

In some embodiments, the target nucleic acid molecule is longer than 200 nt.

In some embodiments, the first number of voltage pulses is 30 or more.

In some embodiments, the first number of voltage pulses may exceed the second number of voltage pulses by 5 or more 0.1.

Another aspect of the present disclosure is a computer product comprising a non-transitory computer readable medium storing a plurality of instructions that when executed control a computer system to perform the aforementioned method and its embodiments.

Another aspect of the present disclosure is a system comprising the computer product, and one or more processors for executing instructions stored on the computer readable medium.

Further aspects of the present disclosure are a system comprising means for performing any of the above methods, a system comprising one or more processors configured to perform any of the above methods, and a system comprising modules that respectively perform the steps of any of the above methods.

A better understanding of the nature and advantages of embodiments of the present invention may be gained with reference to the following detailed description and the accompanying drawings.

### BRIEF DESCRIPTION OF THE DRAWINGS

FIG. 1 shows components of surrogate polymers according to embodiments of the present invention.

FIG. 2 shows XNTP structural detail according to embodiments of the present invention. The molecular structures shown in FIG. 2 may not be chemically correct. FIG. 2 is provided for illustrative purposes.

FIG. 3 illustrates attachment of an extension oligomer to a solid substrate for surrogate polymer synthesis according to embodiments of the present invention.

FIG. 4 illustrates the structure of an extension oligomer, a leader, and nearby components according to embodiments of the present invention. The molecular structures shown in FIG. 4 may not be chemically correct. FIG. 4 is provided for illustrative purposes.

FIG. 5A shows an example of a single molecule-multi-molecular trace event with a typical sequencing by expansion waveform according to embodiments of the present invention.

FIG. 5B illustrates a molecule not clearing a pore according to embodiments of the present invention.

FIG. 6 shows one strategy for sequencing surrogate polymers involving the same forward and reverse voltages according to embodiments of the present invention.

FIG. 7 shows a strategy for sequencing surrogate polymers involving different forward and reverse voltages according to embodiments of the present invention.

FIG. 8 shows a strategy for sequencing surrogate polymers involving a shortened bright period duration according to embodiments of the present invention.

FIG. 9 shows the bright period and dark period schedule with a sequencing strategy according to embodiments of the present invention.

FIG. 10 shows a strategy for sequencing using an inverted nanopore according to embodiments of the present invention.

FIG. 11 illustrates a voltage schedule for processive multi-pass read generation according to embodiments of the present invention.

FIG. 12 illustrates assembled trapped raw read series according to embodiments of the present invention.

FIG. 13 shows meta-periods for clearing surrogate polymers from nanopores according to embodiments of the present invention.

FIG. 14 shows assembled trapped raw read series according to embodiments of the present invention.

FIG. 15A and FIG. 15B show the effect of different concentrations of redox pairs in open channel ADC according to embodiments of the present invention.

FIG. 16 is a flowchart of an example process for sequencing a target nucleic acid molecule according to embodiments of the present invention.

FIG. 17 illustrates a measurement system according to embodiments of the present invention.

FIG. 18 shows a block diagram of an example computer system usable with systems and methods according to embodiments of the present invention.

### DETAILED DESCRIPTION

Embodiments described herein include protocols for nanopore sequencing, including Sequencing By eXpansion (SBX™) protocol. Protocols include a “lead” portion of the molecule that has the property that it enters the pore easily in the forward direction but has a high barrier to translocating through the pore in the reverse direction. Additionally, embodiments include a modified bright and dark period applied voltage pattern that is designed to electrically trap surrogate polymer (e.g., Xpandomer) molecules in the nanopore and position molecules in a controlled way so as to allow for multiple passes or reads on the same surrogate polymer molecule. Furthermore, embodiments include applying higher voltage pulses and/or longer cycle times on periodic bright/dark cycles to finally clear the molecule from the pore. Some embodiments include employing selective application of higher voltage pulses to clear molecules only from specific pores each global pulse application period. Advantages include enrichment of compound molecular trace events (i.e., subreads from the same molecule in a series of bright periods). Other advantages may include increases in pore occupancy and raw base call throughput. Furthermore, protocols may result in higher accuracy multipass reads, as compared to single pass reads. Embodiments may include running with much shorter bright periods without permanently cutting off reads of long surrogate polymer molecules. This may allow for practical experimental conditions that lead to short bright period decay time constants.

Additionally, embodiments may include the ability to dynamically elect to spend more time on surrogate polymers from specific UMI (unique molecular identifier) Molecular Families and spend less time on surrogate polymers from other UMI Molecular Families. UMIs may be added to sample nucleic acids during the sample prep phase. Every nucleic acid fragment from a particular sample will have the same UMI. Different samples will have different UMIs. The UMIs allow for pooling different samples together for sequencing while identifying the sample of each nucleic acid molecule. During sequencing, one may determine that sequences from a particular UMI family (i.e., sample) may need additional sequencing. For example, the confidence of certain base calls may be low due to a relatively high number of different base calls for the same position. Fragments from these samples may be sequenced extra times using the methods described herein.

### I. Sequencing by Expansion

Embodiments may be applied to sequencing by expansion (SBX) using nanopores. Sequencing by expansion is described in WO 2020/236526 A1, “Translocation control elements, reporter codes, and further means for translocation control for use in nanopore sequencing,” filed May 14, 2020, and U.S. Pat. No. 7,939,259 B2, “High throughput nucleic acid sequencing by expansion,” filed Jun. 19, 2008, the entire contents of both of which are incorporated herein by reference for all purposes.

Sequencing by expansion protocol is based on the polymerization of highly modified, non-natural nucleotide analogs referred to as “XNTPs”. In general terms, SBX uses biochemical polymerization to transcribe the sequence of a DNA template onto a measurable polymer called an “Xpandomer.” The transcribed sequence is encoded along the Xpandomer backbone in high signal-to-noise reporters that are separated by ˜10 nm and are designed for high-signal-to-noise, well-differentiated responses. These differences provide significant performance enhancements in sequence read efficiency and accuracy of Xpandomers relative to natural DNA.

### A. Surrogate Polymer Structure

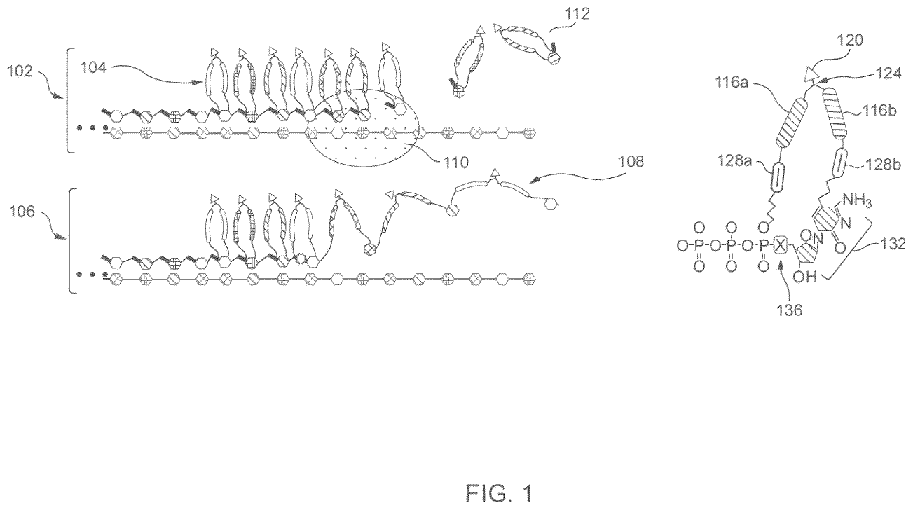

FIG. 1 shows components of surrogate polymers (e.g., Xpandomers). Xpandomers include a sequence of ordered XNTPs which correspond to the order of the target DNA sequence. FIG. 1 is not to scale. XNTPs are expandable, 5′ triphosphate modified non-natural nucleotide analogs compatible with template dependent enzymatic polymerization. An XNTP has two distinct functional regions; namely, a selectively cleavable phosphoramidate bond, linking the 5′ α-phosphate to the nucleobase, and a symmetrically synthesized reporter tether (SSRT) that is attached within the nucleoside triphosphoramidate at positions that allow for controlled expansion by cleavage of the phosphoramidate bond. The SSRT includes linkers and separated by the selectively cleavable phosphoramidate bond. Each linker attaches to one end of a reporter code.

Section 102 shows primer-directed Xpandomer synthesis. XNTP 104 is illustrated in the “constrained configuration”, characteristic of the XNTP substrates and the daughter strand products of template-dependent polymerization. The constrained configuration of polymerized XNTPs is the precursor to the expanded configuration (XNTP 108), as found in Xpandomer products. Section 106 illustrates cleaving to expand the Xpandomer. The transition from the constrained configuration to the expanded configuration occurs upon scission of the P—N bond of the phosphoramidate within the primary backbone of the daughter strand.

During assembly, the monomeric XNTP substrates (XATP, XCTP, XGTP and XTTP) are polymerized on the extendable terminus of a nascent daughter strand by a process of template-directed polymerization using single-stranded template as a guide. Generally, this process is initiated from a primer and proceeds in the 5′ to 3′ direction. Generally, a DNA polymerase 110 or other polymerase is used to form the daughter strand, and conditions are selected so that a complimentary copy of the template strand is obtained. After the daughter strand is synthesized, the coupled SSRTs form the constrained Xpandomer that further forms the daughter strand. SSRTs in the daughter strand have the “constrained configuration” of the XNTP substrates. The constrained configuration of the SSRT is the precursor to the expanded configuration, as found the Xpandomer product.

In this example, once synthesis and expansion are complete, each monomeric XNTP unit 112 in the Xpandomer contains two reporter codes 116a and 116b with a Reporter Code “level” corresponding to the base type it encodes, and a Translocation Control Element (TCE) 120. The TCE controls the rate of Xpandomer translocation through a nanopore through a combination of sterics, electrorepulsion, and/or preferential interaction with the nanopore. The resistance of the TCE to the driving force of the ion current when positioned at the pore aperture and the consequent increase in applied voltage (i.e., the voltage pulse) necessary to overcome the arrest and resume translocation, can be customized by modulating various properties of the TCE, (and in some embodiments, the reporter codes and other elements of the SSRT) e.g., the bulk, length, and/or charge density.

Brancher 124 is the branched structure that terminates the TCE and links to reporter codes 116a and 116b. Enhancers 128a and 128b may aid in polymerase incorporation. Nucleotide 132 is attached to enhancer 128b and may include a cleavable linker 136. Cleavable linker 136 may be a photocleavable linker. Cleavable linker 136 may be cleaved to result in the expansion shown in section 106.

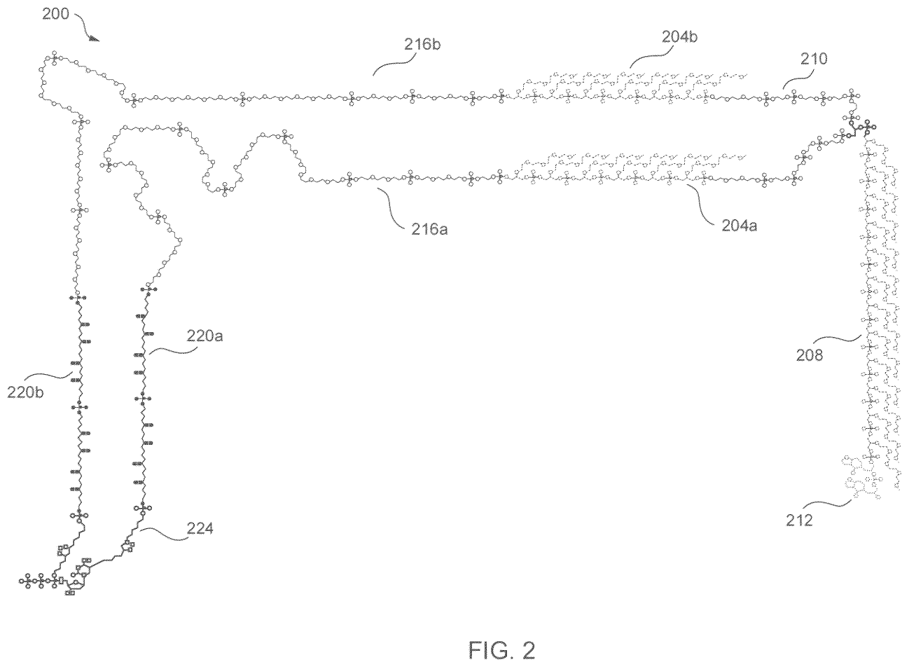

FIG. 2 shows structural details of XNTP 200. In FIG. 2, the XNTP includes two reporter codes 204a and 204b. and a translocation control element (TCE) 208. Different reporter codes are sized to block ion flow through a nanopore at different measurable levels. Reporter codes and other features can be designed by selecting a sequence of specific phosphoramidites.

In certain embodiments, TCEs are polymers produced by solid-phase synthesis using the phosphoramidite method with suitable monomeric building blocks that terminate with a branched structure (i.e., the “brancher”). Branched phosphoramidites include both symmetrical and asymmetrical branchers. In one embodiment, the TCE brancher 210 is a symmetrical branching CED phosphoramidite, wherein each arm of the brancher is linked to a reporter code. Exemplary symmetrical chemical branchers include 1,2,3-O-tris-(phosphosphodiester)propane, 1,3-bis-(5-O-phosphodiester-pentylamido)-2-O-phosphodiester-propane, and 1,4,7-O-tris-(phosphodiester)-heptane.

A UV chromophore 212 may be attached to the end of TCE 208. UV chromophore 212 may allow for visualization or quantification. Spacers 216a and 216b are attached to reporter codes 204a and 204b, respectively. Spacers 216a and 216b may be polyethylene glycol (PEG) units, which may modulate the length traversed in a pore. Enhancers 220a and 220b may be attached to spacers 216a and 216b. Enhancers 220a and 220b may be positively charged spermine that facilitates polymerase incorporation. Nucleotide 224 may be attached to enhancers 220a and 220b. Nucleotide 224 may include a triphosphoramidate diester. The structural elements of XNTP 200 may be adjusted for improving measurement within a nanopore.

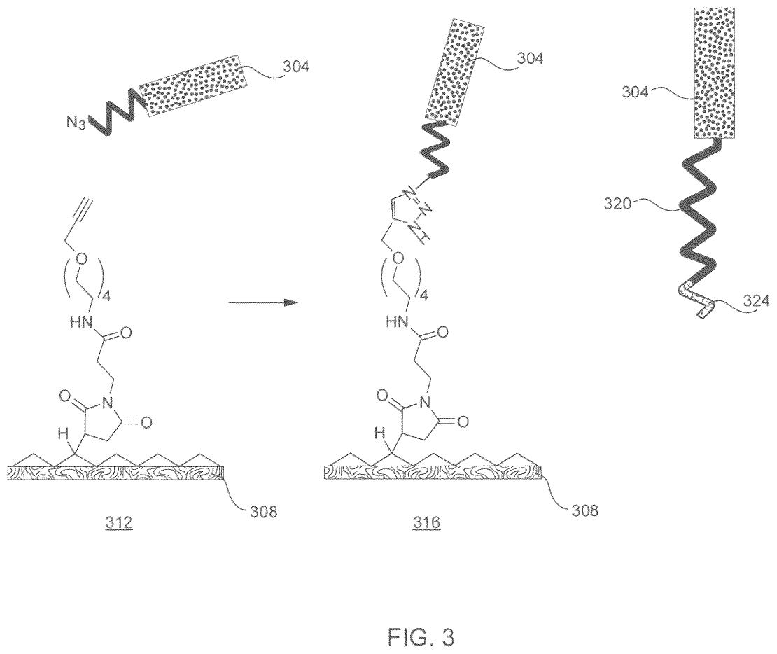

FIG. 3 illustrates attachment of an extension oligomer 304 to a solid substrate 308 to prepare for Xpandomer synthesis. Solid substrate 308 may be crosslinked to maleimide. Stage 312 shows extended oligomer 304 not attached to solid substrate 308. After click chemistry at stage 316, extended oligomer 304 is attached to solid substrate 308. Extended oligomer 304 may be attached to a leader portion 320. Leader portion 320 may be attached to a photocleavable linker 324.

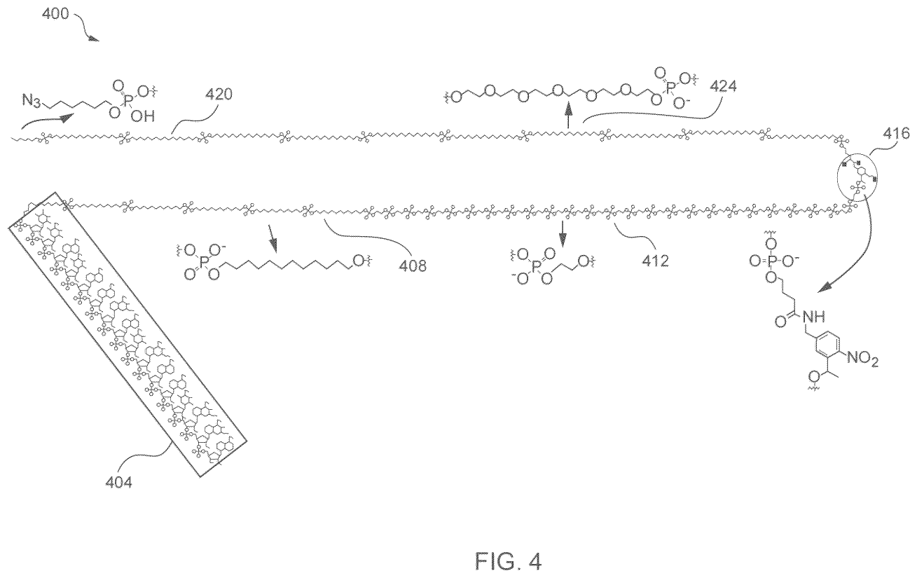

FIG. 4 illustrates structure 400 of extension oligo 404, “leader” (L), and nearby components. Extension oligo may be a 2′ O-methyl extension oligo. Adjacent to the extension oligo 404 is Poly-C12 Spacer (Z) 408, followed by Poly-C2 Spacer (L) 412. Photocleavable spacer (PC) 416 and segments beyond (i.e., azide (R) 420 and spacer (D) 424) are not part of the Xpandomer in the sequencing protocols described herein, because photocleavage occurs just after Xpandomer synthesis and prior to loading any xpandomer on the sequencer. Azide R 420 may be a 5′-azide. Spacer D 424 may be a poly-PEG6 spacer. A shorthand sequence for structure 400 may be R(D)₁₀(PC)(L)₂₅(Z)₆(TCATAAGACGAACGGA), where the sequence at the end represents extension oligo 404. During some Xpandomer sequencing operations after the flow of the xpandomer molecules into the fluidic channel above the membrane, the hydrophobic Poly-C12 Spacer may partition onto the membrane phase, initiating quasi 2D diffusion/migration along the surface of the membrane. 3D diffusion/migration and fluid flow may also a play role in mass transport of Xpandomers towards the pore. Xpandomer capture is believed to occur when the highly negatively charged Poly-C2 Spacer segment (L) is electrostatically drawn into the barrel of the nanopore during application of a “Positive” applied bias across the membrane. Note that for inverted pores, the bottom of the barrel of the pore protrudes slightly from the bilayer membrane on the Cis side.

### B. Operation

During the “bright period,” Xpandomer molecules capture and begin to translocate through the nanopore due to a combination of both baseline and TCE applied voltage pulses. Baseline voltages are sufficient to read the tag code at each XNTP position, and the short, higher voltage TCE pulses are designed to overcome the energetic barrier associated with a TCE. Ideally, each TCE pulse results in translocation past a single TCE barrier, thus moving the Xpandomer further into the pore in the forward direction by an amount of one “base” position.

During typical operation, applied voltage patterns are designed so that there are a fixed number of TCE pulses during each bright period, which cause the Xpandomer to translate in the “forward” direction by a number of bases, corresponding to the number of TCE pulses, or until the Xpandomer fully translocates, and is released into the fluidic “trans” chamber below the membrane.

During typical operation, an Xpandomer molecule may not fully translocate prior to the end of a single bright period. This may happen due to the molecule capturing late in the bright period and having an Xpandomer length with more base positions than there are TCE pulses remaining in the bright period. A molecule can get stuck while attempting to translocate in the forward direction for a variety of reasons. There may be a base position which has a defect (such as a failed cleavage event) which makes it impossible or very difficult for the molecule to translocate past that point. In such circumstances, and for other reasons, an Xpandomer may not be able to fully translocate during the bright period, regardless of the number of TCE pulses in a bright period. In such situations, it may be observed that a number of base positions in the beginning of the read are sequenced and generate the expected signal levels, until the defective position is reached. The last tag code level located just before the defect can then be observed for the remainder of the bright period. In order that pores do not remain permanently clogged, a large negative voltage may be applied for some fraction of time in the dark period in order to clear out any stuck molecules by driving them hard in the reverse direction.

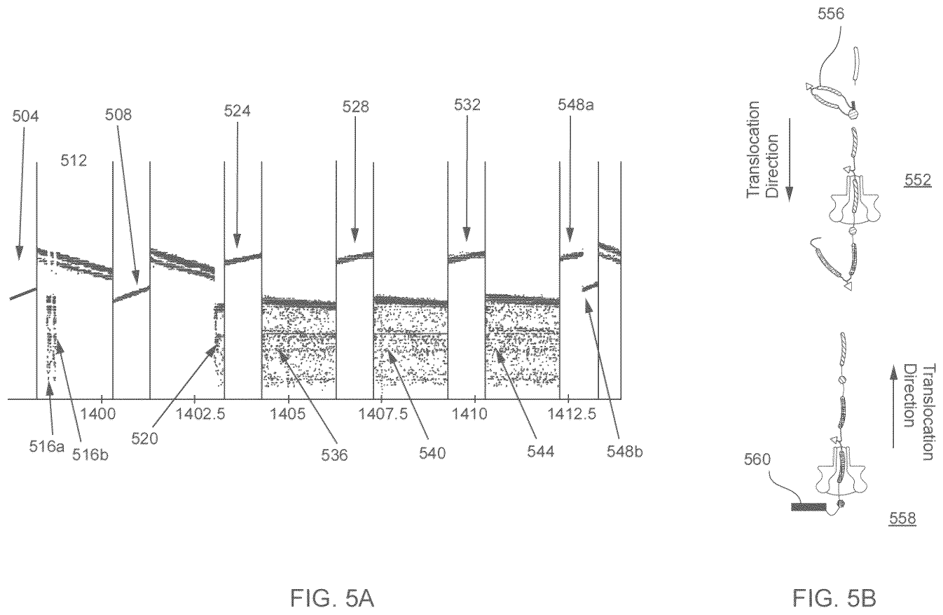

FIG. 5A shows an example of a single molecule-multi-molecular trace (SM3T) event with a typical sequencing by expansion waveform. The graph shows time in seconds on the x-axis. The graph shows voltage readings on the y-axis. Other electrical measurements may be used instead of voltage, including voltage equivalents (e.g., ADC counts) or current. Dark periods 504 and 508 are normal dark periods, where the pore is clear. Bright period 512 shows signals 516a and 516b of molecule 1 and molecule 2, respectively.

Signal 520 shows molecule 3 during a bright period. The event shows that molecule 3 gets stuck in the pore and does not clear over several cycles (dark periods 524, 528, 532 and signals 536, 540, and 544 in bright periods). Eventually, the molecule clears in a dark cycle, as indicated by the change from signal 548a to signal 548b (when molecule 3 clears). This event may result from properties of the Xpandomer's leader segment, which create difficulty in the leader translocating in the reverse direction.

FIG. 5B illustrates a possible mechanism behind the trace in FIG. 5A. Diagram 552 shows a bright period. The translocation direction is downward. Uncleaved position 556 would hit the pore after the next pulse. Normal tag code level is expected. Diagram 558 shows a dark period. The translocation direction is now upward. Leader 560 has trouble translocating in the reverse direction (upward) through the pore. Eventually, leader 560 goes through the pore.

Xpandomer molecules can be designed with properties in the leader portion of the Xpandomer that cause the leader to behave differently in the forward and reverse directions. During the bright period (forward direction), the leader may have characteristics that allow the leader to be captured into the pore from the cis side with relatively high capture rates under reasonably applied voltages. Following capture, but still during the same bright period, the leader may protrude from the underside of the pore (trans side of membrane), as TCE pulses cause the molecule to process steadily through the pore.

During the dark period (reverse direction), if a molecule is still in the pore when a dark period begins, the molecule should begin to translocate in the reverse direction under a negative applied voltage. Once the Xpandomer molecule has almost fully reversed its position (i.e., it has almost fully backed out), the leader may remain on the trans side of the barrel. At this point, the desired property of the leader is that the leader has a high energetic barrier to entry into the barrel from the trans side, and thus is highly resistant to translocation through the barrel in the trans to cis direction (forward direction).

### II. Strategies for Sequencing

Strategies for leveraging asymmetric leader behavior (with respect to direction through the pore) are described herein. These strategies may increase accuracy through reducing the number of Xpandomers that get stuck in a pore.

A. Full Forward and Full Reverse with Same Voltage

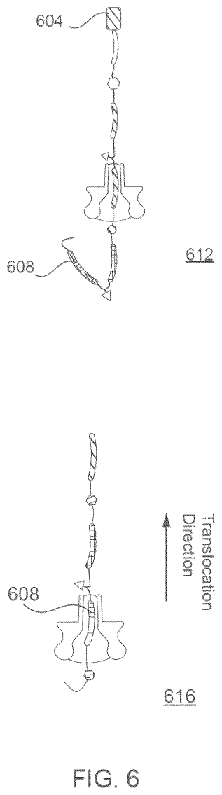

FIG. 6 shows one strategy for sequencing Xpandomers involving the same forward and reverse voltages. FIG. 6 shows a nanopore with a membrane below the nanopore. An Xpandomer has a blocker 604 (e.g., streptavidin) on one end and a leader 608 on the other end. The leader threads through the pore (in the downward direction in the figure). The Xpandomer moves downward and is sequenced during bright periods (e.g., diagram 612) until the end of the molecule and/or the end of the bright period. Voltage is applied to move the Xpandomer downward. Voltage includes a baseline voltage to move the Xpandomer downward so that the reporter elements can be read. To move the Xpandomer past a TCE element, a higher voltage is applied. Blocker 604 prevents the Xpandomer from exiting the pore.

During the dark period (e.g, diagram 616), the applied voltage is reversed, and the Xpandomer translocates in the upward direction. The same voltage is applied in the dark period as the bright period, but the voltage has an opposite polarity. Leader 608 may have difficulty going through the membrane and/or the pore in the opposite direction.

The distribution of SM3T durations may be exponential. The distribution may show many shorter lengths, reflecting Xpandomers getting stuck in the nanopore. The mode of durations should equal 1.

B Full Forward and Full Reverse with Increased Reverse Voltage

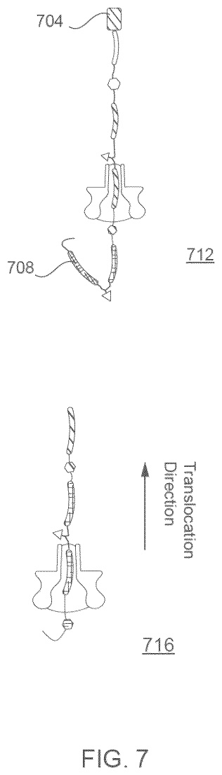

FIG. 7 shows a strategy for sequencing Xpandomers involving different forward and reverse voltages. The nanopore and Xpandomer are configured similar to FIG. 6. The Xpandomer has a blocker 704 on one end and a leader 708 on the other end. Bright period in diagram 712 may be the same as bright period in diagram 612. During the dark periods, the leader may be stuck and may not be able to go through the nanopore. To address this situation, the reverse voltage is increased for some periodic dark cycles (e.g., dark period in diagram 716). For example, one out of every 10 or one out of every 20 dark cycles may include an increased reverse voltage to dislodge any stuck leaders. This increased voltage may be called a “clearing voltage.”

The shape of the distribution of SM3T durations may be changed from the distribution with FIG. 6. Fewer Xpandomers get stuck in the nanopore, so the distribution shows longer lengths. The mode may not be equal to 1.

### C. Limited Bright Period Duration

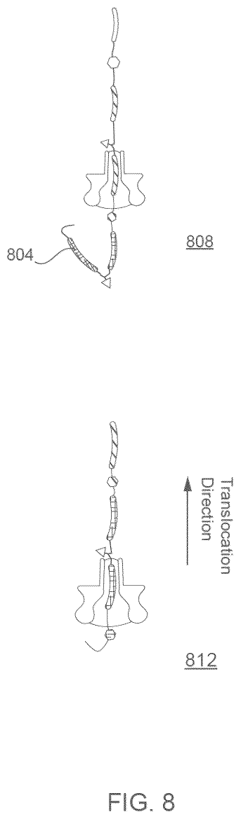

FIG. 8 shows a strategy for sequencing Xpandomers involving a shortened bright period duration. FIG. 8 shows a nanopore with a membrane below the nanopore. An Xpandomer has a leader 804 on the other end and no blocker on the other end. Leader 804 threads through the pore (in the downward direction in the figure). The Xpandomer moves downward and is sequenced during bright periods (e.g., diagram 808). The bright periods are shortened with the intent to prevent certain size Xpandomers from traveling through and exiting the nanopore in the forward direction. Shorter Xpandomers may fully translocate through and escape. Each bright period may include a fixed, predetermined number of TCE voltage pulses. A fixed number of bright periods may be applied. A dark period (e.g., diagram 812) is then applied to reverse the direction of the Xpandomer. Dark period in diagram 812 may be the same as dark period in diagram 616.

The shape of the distribution of SM3T durations may be changed from the distribution with FIG. 6. The mode may not be equal to 1. The distribution may collapse read length distribution to most reads in mode +/− a few bases.

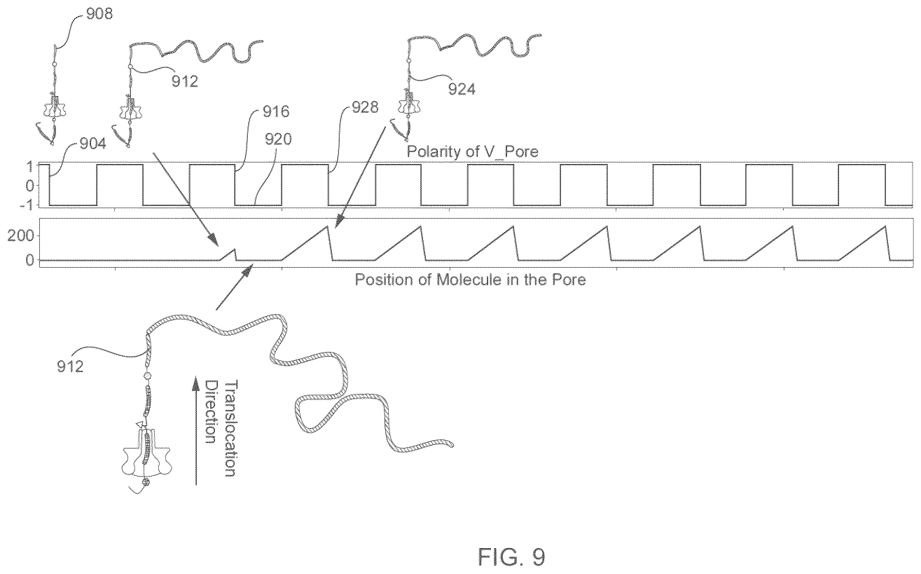

FIG. 9 shows the bright period and dark period schedule with a sequencing strategy. The top graph shows the polarity of the voltage applied across a nanopore. During the bright period, the polarity is shown as 1. During the dark period, the polarity is shown as −1. The top graph does not show the magnitude of voltage, only the polarity. The bottom graph shows the position of the molecule in the pore. A position of 1 corresponds to the unit of the Xpandomer closest to the leader. A high position number (e.g., 200) corresponds to a unit of the Xpandomer far away from the leader and closer to the opposite end of the Xpandomer in this example.

As shown in FIG. 9, bright period 904 may include normal sequencing of Xpandomer 908. Sequencing may be limited by the bright period duration or full translocation and escape. Xpandomer 908 and all Xpandomers in FIG. 9 do not have a blocker on the tail end of the molecule.

An Xpandomer 912 may enter the nanopore late in bright period 916. There may be more bases (e.g., reporter elements) remaining after the bright period is over. With the dark period, Xpandomer 912 moves completely out of the nanopore.

In FIG. 9, Xpandomer 912 has more units (e.g., TCEs) than the number of pulses in the bright period. During dark period 920, a high voltage is applied to ensure the leader reaches the pore. A lower voltage is applied after that. The particular Xpandomer never fully translocates through the nanopore. The duration of the bright period may be increased so that more of the Xpandomer goes through the nanopore. However, too long of a bright period may result in Xpandomers translocating through the nanopore in only one direction. Additionally, different sized Xpandomers may be present in the sample. A certain bright period duration may result in shorter Xpandomers translocating through the nanopore in only one direction while also not being sufficient to sequence all XNTPs in longer Xpandomers.

Xpandomer 924 may arrive in the pore early in bright period 928. The molecule may have more bases than there are pulses remaining in the bright period. Thus, Xpandomer 924 does not fully translocate through. Almost exactly as many bases are read as there are pulses in the bright period.

### D. Inverted Nanopore

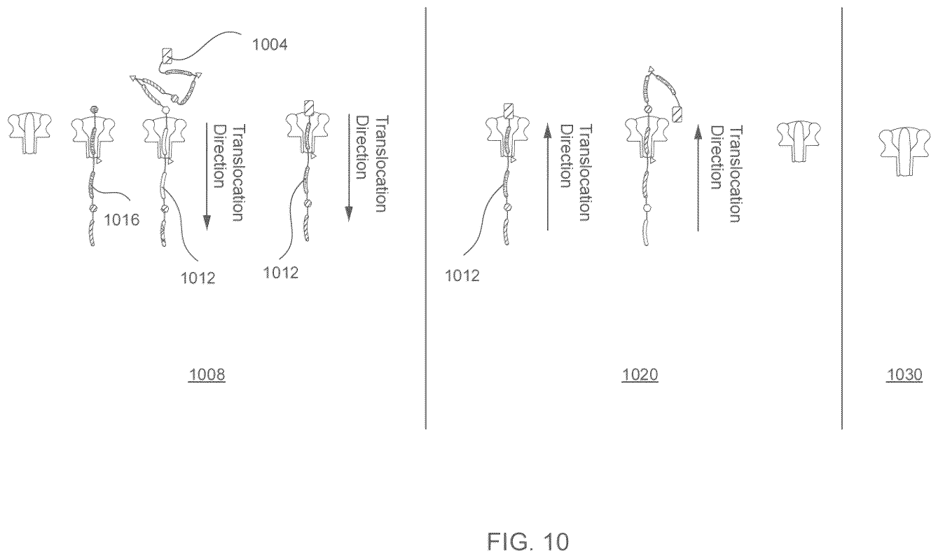

FIG. 10 shows a strategy for sequencing using an inverted nanopore. Pores are inserted form the cis side, as has historically been done for SBT on HTP. The Xpandomer includes a leader at one end and a blocker (e.g., blocker 1004) at the other end. The leader enters the membrane before the nanopore. The Xpandomer is sequenced while reversing the Xpandomer out of the nanopore.

In step 1008, the Xpandomer is captured. A high voltage (e.g., TCE voltage) is applied for a longer period of time (e.g., 0.1 to 10 s of milliseconds instead of 8 μs). A captured molecule 1012 with a blocker moves to an end so that further translocation is stopped by the blocker. Also shown in step 1008 is a molecule 1016 with no blocker that is captured. Molecule 1016 may include fragmented molecules. The molecule with no blocker may quickly pass through the nanopore.

In step 1020, Xpandomer 1012 is moved in the reverse direction to back out Xpandomer 1012 from the nanopore. The TCE voltages are applied in the reverse direction. Sequencing information is obtained as the molecule is backing out. All captured molecules are expected to be positioned at the molecule's end (or at an uncleaved position). Molecules are pulsed in the reverse direction and data is acquired as the molecule is being backed out. The bright periods may be as long as the longest expected molecules in the sample. For example, for a ctDNA assay, the longest expected molecules may be 350 bp, corresponding to 350 pulses or 350 ms for 1 ms interpulse durations.

In step 1030, an optional recharge may be performed before the next capture step. A dark period may be applied. The dark voltage may help recharge the electrode if the cumulative voltages from steps 1 and 2 are not balanced. During this period, no data is acquired. If phased array mode is being run, then this dark period might be the duration of steps 1008 and 1020, allowing for the other half of the chip (phase) to complete those two steps.

The leader may be modified so that it does not translocate easily when moving through the pore starting on the vestibule (i.e., membrane) side. Xpandomers may need to be captured reasonably well when entering from the vestibule side. High voltage may help capture the Xpandomers. Membranes may be resilient to high voltage for longer times than with other strategies (e.g., with 8 μs pulse durations). A block may be added to the end of the Xpandomer.

The benefits of this strategy may include filtering out a fraction of fragmented molecules. Additionally, the read starts may be synchronized. Furthermore, pore insertion may be from the cis side. For example, a well may be filled with a nanopore solution. The well then can be covered with a membrane, and the nanopore may be inserted from the cis side.

### III. Processive Consensus Strategies

Strategies may include reading the same XNTPs multiple times in a nanopore and applying voltages to clear stuck Xpandomers. These strategies may increase accuracy through reducing the number of Xpandomers that get stuck in a pore and/or through repeating reads of XNTPs through the nanopore.

### A. Clearing/Recharge Voltage Applied to All Cells

A strategy for sequencing a molecule may not involve reading a molecule all the way from start to end each pass. A part of the molecule may move through the nanopore for a subread, rather than the whole molecule moving through the nanopore for a complete read. Additionally, the molecule may move forward and then backward to make many short overlapping passes and a processive way. Bright and dark period durations may be shortened compared to other strategies. The number of high voltage TCE pulses may be less than the number of TCEs in an Xpandomer. The number of TCE pulses in bright periods would be greater than the number of reverse TCE pulses in dark periods.

As an example, the Xpandomer length distribution may have a peak around 350 bp. The bright period may include a duration of 30 TCE pulses. The dark period duration may equal in total time to bright period duration but with an applied voltage pattern that contains 25 TCE pulses in the reverse direction. Having equal durations of bright periods and dark periods allow balancing charge on the electrode, regenerating and/or resetting the electrode. The protocol may include additional periods that include “clearing voltages” to periodically remove the occasional stuck Xpandomer.

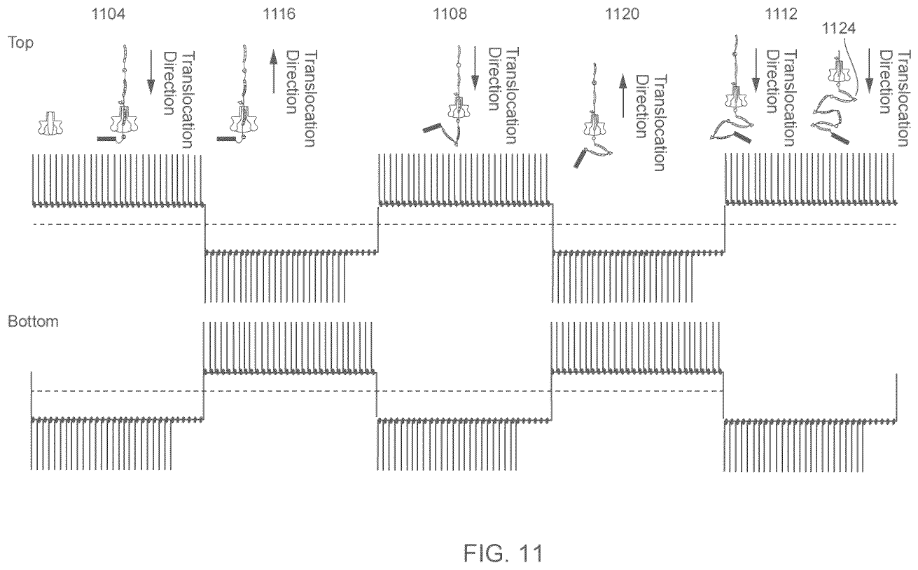

FIG. 11 illustrates a voltage schedule for processive multi-pass read generation. The graph shows voltage pulses for the top electrode and for the bottom electrode. The dashed lines indicate a voltage of 0 polarity for each respective electrode. Voltages above the dashed line have a positive polarity. Voltages below the dashed line have a negative polarity. During the bright periods, the top electrode has pulses of a positive polarity, and the bottom electrode has pulses of a negative polarity. Bright periods 1104, 1108, and 1112 are shown. During the dark periods, the top electrode has pulses of a negative polarity, and the bottom electrode has pulses of a positive polarity. Dark periods 1116 and 1120 are shown. The dark period is illustrated as having five fewer pulses as the bright period.

During the bright period, the Xpandomer moves in the downward direction as illustrated. The Xpandomer is sequenced during the bright period. Each pulse should correspond to a read. Capture of the Xpandomer may occur at any time during a bright period. During the dark period, the Xpandomer moves in the reverse (upward) direction. No sequencing is performed during the reverse direction. If the leader backs into the vestibule, the Xpandomer may get stuck. The meta-periods with clearing voltages may dislodge the Xpandomer. The meta-periods are not shown in FIG. 11.

After a sufficient number of cycles, a molecule (e.g., Xpandomer 1124) exits the pore in the forward direction.

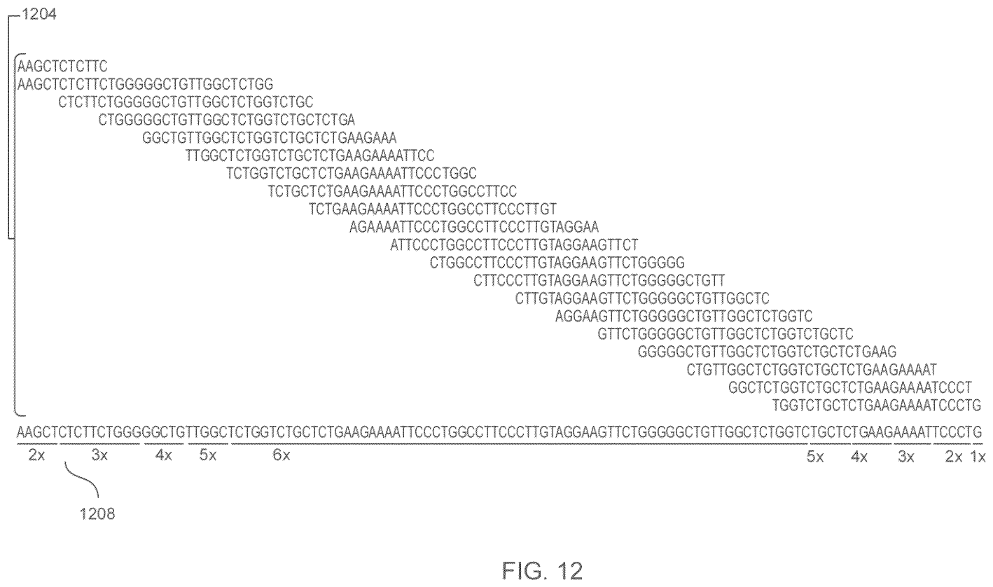

FIG. 12 is an illustration of the assembled trapped raw read series resulting from a protocol similar to that used in FIG. 11. The Xpandomer length corresponds to a target nucleic acid molecule that is 116 bp long. The Xpandomer was moved with forward cycles of 30 pulses and reverse cycles of 25 pulses. In total, 20 cycles were used to cover the entire length of the Xpandomer. The reads for each cycle are shown in section 1204. Because each cycle includes reads that overlap, individual nucleotides are sequenced several times. The trapped consensus read is shown at read 1208. Underneath the trapped consensus read shows the number of times the nucleotide has been sequenced. For example, the initial subsequence of AAGCT is sequenced twice. The middle section starting with TCTGGT is sequenced six times. The beginning of the Xpandomer can be sequenced multiple times if the initial forward and reverse cycles were set to have the same number of pulses before changing to cycles where the bright period has more forward pulses than the dark period has reverse pulses. The end of the Xpandomer can be sequenced multiple times by continuing the forward and reverse pulses until the Xpandomer has fully exited the nanopore.

As an example, each half cycle may be 30 ms at 1 ms interpulse spacing. A full cycle may take 60 ms. The total time spent on a molecule may be 1200 ms. A total of 577 raw bases may be read in 1.2 s total time or 0.6 s bright time. With the exception of 20 bases at either end, the 116 bp molecule is sequenced at 6× depth of coverage.

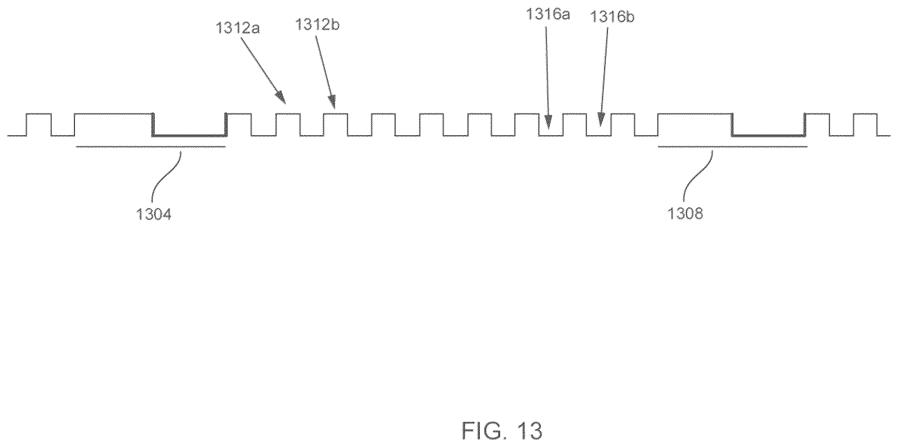

FIG. 13 shows meta-periods for clearing Xpandomers from nanopores. The meta-periods themselves may contain a number of AC periods and a bright and dark clearing cycle. The AC periods may be similar to those typically used in sequencing except shorter than 1 or 2 seconds. The pulses that are indicated by segments 1304 and 1308 are the bright and dark clearing cycles. The other pulses have normal voltages (i.e., same as TCE voltage pulses during the non-meta-periods) for bright periods and dark periods. The number of pulses in bright may be set at a number to clear the Xpandomer if the leader did not get stuck. For example, the number of pulses in the bright period may equal to the median number of TCE units in the Xpandomer length distribution. The dark periods may be sufficient to drive the Xpandomer back to the reverse position.

A special bright period and dark period may be executed once, twice, or more each meta-period. The special bright and dark periods may have higher applied voltage and/or be a longer duration. In FIG. 13, the special period is three times the duration of a normal cycle of one bright period and one dark period. The entire meta-period is 10 AC periods long. The meta-periods may be applied every 10, 15, or 20 AC periods. In FIG. 13, the special period is 3×, and the meta-period is 10 AC periods long.

Normal bright periods (e.g., bright periods 1312a and 1312b) may have an AC modified period much shorter than 1 or 2 seconds. The number of pulses in the bright period may be equal to the median of the input xmer fragment length distribution. Normal dark periods (e.g., period 1316a and period 1316b) may have a high voltage initially in the period to quickly drive the molecule in reverse back to the leader position.

### B. Clearing/Recharge Voltages Applied to Specific Cells

The protocol of processive consensus described with FIGS. 11-13 may be modified. The meta-periods and the clearing/recharge voltages may be applied to only cells with stuck Xpandomers. The periods may be applied every first, second, third, fourth, or fifth AC period instead of every 10^{th} or 20^{th} AC period.

By the end of the dark period prior to each meta-period (i.e., clearing period), a decision is made for every active cell in the array on whether the clearing period should be experienced by each cell or whether the cell should be temporarily deactivated during assertion of the global clearing period. A deactivation mask may be updated in preparation for the clearing period.

During the bright and dark clearing periods, the deactivation mask is applied, and high positive and negative voltages are applied to global chip lines. Many or most cells may be temporarily deactivated and thus electrically isolated such that they do not experience a clearing voltage in either the bright or dark clearing period, or both.

In one embodiment, ˜1 ms may elapse to update the deactivation mask for all cells on the Nanopore Sequencer chip. The mask update time may be followed by the bright and/or dark clearing periods. The clearing period itself may last anywhere from one millisecond to tens of milliseconds. Following the clearing period, another ˜1 ms duration of time may be allocated to re-activate cells with a second deactivation mask update.

### C. Advantages of Processive Consensus

Both processive consensus applied to all cells and applied to specific cells may include individual bright periods that are much shorter than what is required to traverse the full length of target molecules. Additionally, the subreads produced in consecutive bright periods may overlap at the ends. This leads to a protocol where molecules of any length can be sequenced at their full length with a depth of greater than one. This method is an improvement over the strategy described with FIG. 9, where the bright period length would need to be tuned for a particular input DNA fragment length distribution. Molecules equal to or greater than a certain length would be subject to multiple passes because the molecule would not clear after a cycle of bright and dark periods. The maximum read length on a molecule may therefore be equal to the number of TCE pulses in the bright period. Thus, the ends of molecules longer than that critical length would not be sequenced.

### D. Reducing Overlap

In some embodiments, reducing the number of times the sequences are read may be desired. For example, molecules may be read at single-depth coverage or slightly greater (e.g., an average depth of between 1 and 2). To achieve this, the dark period can be set so there are few TCE pulses in the reverse direction compared to the TCE pulses in the bright period in the forward direction.

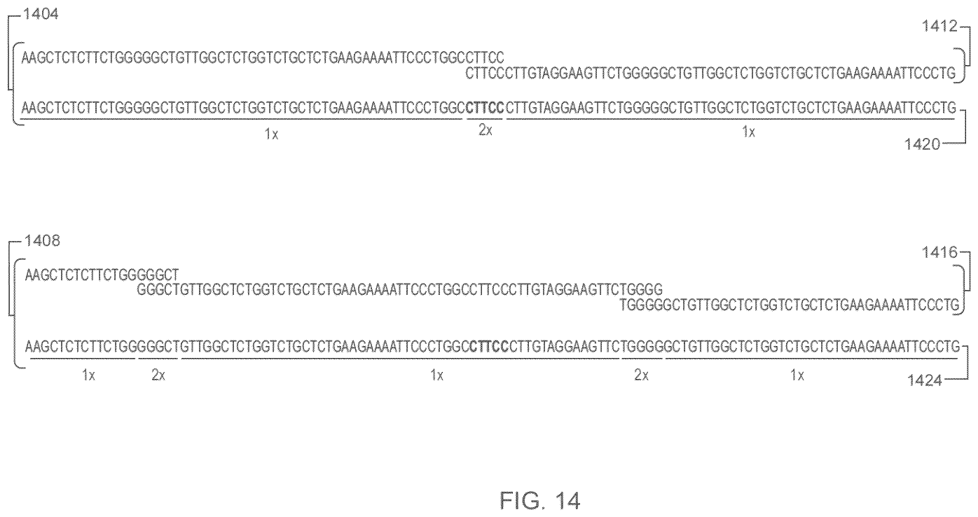

FIG. 14 shows Example Molecule 1404 and Example Molecule 1408 of trapped raw reads when reducing the number of reverse TCE pulses. Reads 1412 and reads 1416 show the series of trapped raw reads. Reads 1420 and reads 1424 show the trapped consensus read with a “single pass limit”. In both examples shown in FIG. 14, the number of reverse TCE pulses is 5. In Example Molecule 1404, the number of forward TCE pulses is 60. In Example Molecule 1408, the number of forward TCE pulses is 65. Most of the nucleotides are read with a single pass, achieving a “single pass limit.” The nucleotides corresponding to the reverse pulses are the ones read in two passes. Increasing the number of forward TCE pulses and/or reducing the number of reverse TCE pulses increase the proportion of nucleotides being read at only a single depth.

Aiming for a depth coverage slightly greater than 1 based on the processive consensus voltage pattern results in a shorter bright period duration than simply sequencing an entire molecule in one single bright period.

### E. Shorter Bright Periods

Short AC modulation periods (i.e., bright periods) result in electrochemical and circuit related advantages. For example, because of the way sequencing is performed with a wet analog circuit, voltage across the membrane/pore decays during a bright period. Dark periods are used to “recharge” both an electrochemical battery in the volume of the well and an electrostatic working electrode capacitor at the bottom of the well. The rate of this bright period decay is governed in part by the size of the working electrode capacitor and also in part by the concentration and volume of electrochemically active redox species within the well. There are some practical advantages to lowering the concentration of electrochemically active redox species used during sequencing, but doing so results in a shorter bright period decay time constant. One way to deal with a shorter bright period decay time constant is simply to shorten the duration of the bright period itself. Processive consensus allows for shortening the bright period duration substantially while not permanently cutting off reads for Xpandomer molecules with lengths longer than the number of TCE pulses in the bright period.

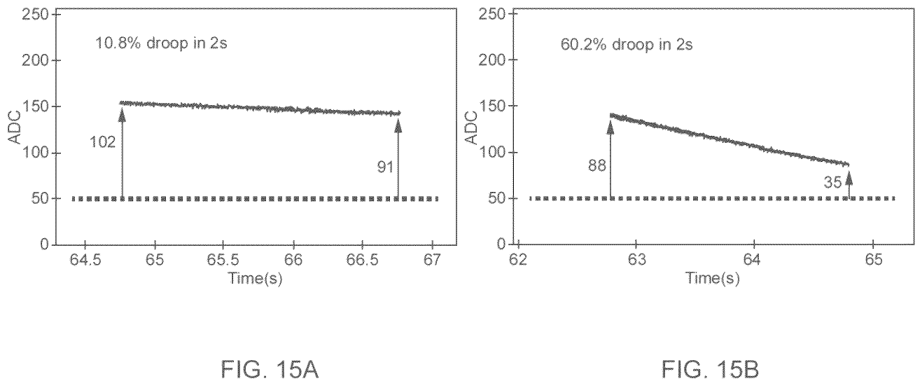

FIG. 15A and FIG. 15B show the effect of different concentrations of redox pairs in open channel ADC. The graphs show time in seconds on the x-axis, and open channel ADC at the start and end of bright periods. FIG. 15A has a high concentration of redox agent. FIG. 15B has aconcentration of redox agent that is eight times lower than FIG. 15A. For FIG. 15A, the difference in the open channel ADC for start and end is 10.8%. For FIG. 15B, the difference between open channel ADC for start and end is 60.2%. FIGS. 15A and 15B show that a lower concentration results in a shorter bright period decay time constant.

### F. Example Methods

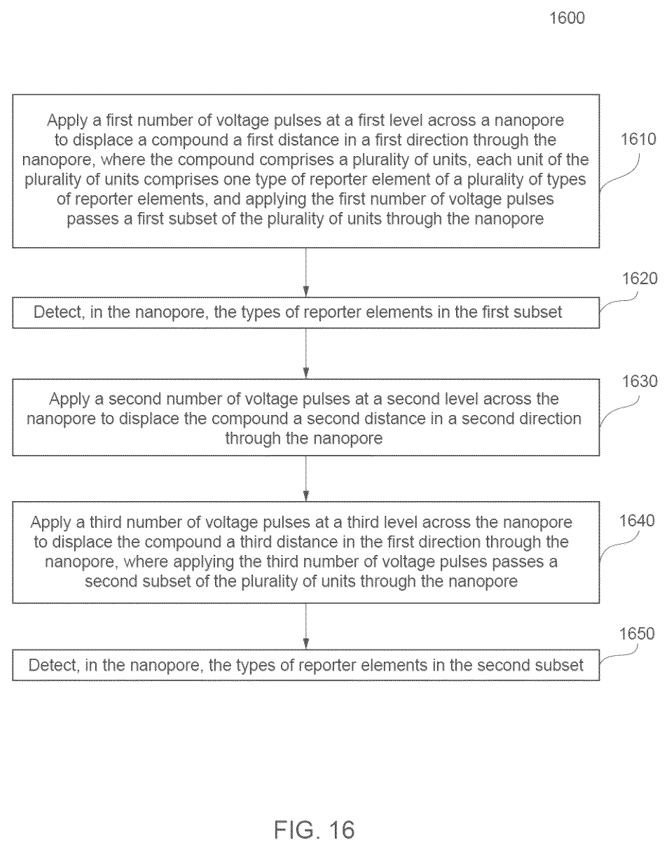

FIG. 16 is a flowchart of an example process 1600 for sequencing a target nucleic acid molecule. In some implementations, one or more process blocks of FIG. 16 may be performed by system 1700, including detector 1702 and logic system 1703. The target nucleic acid molecule may be 100 to 150 nt, 150 to 200 nt, 200 to 300 nt, 300 to 400 nt, or more than 500 nt long.

At block 1610, a first number of voltage pulses at a first level may be applied across a nanopore to displace a compound a first distance in a first direction through the nanopore. The compound may be created from the target nucleic acid molecule. The compound may be a surrogate polymer or an Xpandomer, as described herein and in WO 2020/236526 A1 and U.S. Pat. No. 7,939,259 B2, the entire contents of both of which are incorporated herein by reference for all purposes. Prior to applying the first number of voltage pulses, the first compound may be captured by the nanopore. The compound may include a plurality of units. A unit may be similar to the unit shown in FIG. 1. Each unit of the plurality of units may include one type of reporter element of a plurality of types of reporter elements (also called reporter codes). Each type of reporter element may correspond to an identity of a nucleotide in the target nucleic acid molecule.

The first direction may be in the same direction that the compound moved through the nanopore when the compound was initially captured. For example, the compound may have a leader portion. The leader portion may be captured in the nanopore. The first direction may be in a way where the leader moves farther away from the nanopore.

Applying the first number of voltage pulses may pass a first subset of the plurality of units through the nanopore. The first number of voltage pulses may be the voltage pulses in the bright period. The number of voltage pulses may correspond to the number of units in the plurality of units passed through the nanopore. Each unit of the plurality of units may include a translocation control element. Applying the first number of voltage pulses may pass a first number of translocation control elements through the nanopore. The first number of voltage pulses may equal to the first number of translocation control elements.

The first subset of the plurality of units may include 10 to 20, 20 to 30, 30 to 40, 40 to 50, 50 to 60, 60 to 70, 70 to 80, 80 to 90, 90 to 100, or more than 100 units. The first number of voltage pulses may include 10 to 20, 20 to 30, 30 to 40, 40 to 50, 50 to 60, 60 to 70, 70 to 80, 80 to 90, 90 to 100, or more than 100 pulses.

A baseline level of voltage across the nanopore may be applied to move the units through the nanopore for sequencing in between pulses of the first number of voltage pulses. This lower level of voltage may move the units through the nanopore but may not be enough to move the translocation control element through the nanopore. A voltage at a baseline level may be applied across the nanopore to displace the compound a distance in the first direction through the nanopore. The voltage at the baseline level may be the same polarity as the voltage pulses of the first number of voltage pulses. The baseline level may be less than the first level. The compound after being displaced the distance may have a translocation control element in the nanopore.

As such, in an example, at block 1610, the following actions may be taken: Apply a first number of voltage pulses at a first level across a nanopore to displace a compound a first distance in a first direction through the nanopore, where the compound comprises a plurality of units, each unit of the plurality of units comprises one type of reporter element of a plurality of types of reporter elements, and applying the first number of voltage pulses passes a first subset of the plurality of units through the nanopore.

At block 1620, the types of reporter elements in the first subset may be detected. Signal values may be measured for a nanopore having a voltage applied across the nanopore when reporter elements in the first subset of the plurality of units are in the nanopore. The types of reporter elements in the first subset are determined using the signal values. As a result of the correspondence of the types of reporter elements and the nucleotides, the identities of the nucleotides in the target nucleic acid molecules are also determined.

As such, in an example, at block 1620, the following actions may be taken: Detect, in the nanopore, the types of reporter elements in the first subset.

At block 1630, a second number of voltage pulses at a second level may be applied across the nanopore to displace the compound a second distance in a second direction through the nanopore. The second number of voltage pulses may be voltage pulses in a dark period. The first direction is opposite the second direction. The voltage pulses of the first number of voltage pulses have an opposite polarity as the voltage pulses of the second number of voltage pulses. The second distance may be less than the first distance. The second number may be less than the first number.

The first number of voltage pulses may exceed the second number of voltage pulses by 1, 2, 3, 4, 5, 6, 7, 8, 9, 10 to 15, 15 to 20, 20 to 25, 25 to 30, 30 to 40, 40 to 50, or greater than 50. In some embodiments, the first number of voltage pulses may equal the second number of voltage pulses. Subsequent cycles of pulses may then be unequal so that the pulses end up advancing the compound through the nanopore.

As such, in an example, at block 1630, the following actions may be taken: Apply a second number of voltage pulses at a second level across the nanopore to displace the compound a second distance in a second direction through the nanopore.

At block 1640, a third number of voltage pulses at a third level may be applied across the nanopore to displace the compound a third distance in the first direction through the nanopore. The third number of voltage pulses may be voltage pulses in another bright period. Applying the third number of voltage pulses passes a second subset of the plurality of units through the nanopore. The second subset and the first subset may include some of the same units. The second subset may include units not in the first subset. The third distance may be greater than the second distance. The third level may equal the first level. The third number of voltage pulses may be the same or different as the first number of voltage pulses.

As such, in an example, at block 1640, the following actions may be taken: Apply a third number of voltage pulses at a third level across the nanopore to displace the compound a third distance in the first direction through the nanopore, where applying the third number of voltage pulses passes a second subset of the plurality of units through the nanopore.

At block 1650, the types of reporter elements in the second subset may be detected. A sequence of the target nucleic acid molecule may be determined. The sequence may be determined from the order of the types of reporter elements detected. For one or more units in both the first subset and the second subset, the same type of reporter element may be detected. For example, the same reporter elements may be detected as described in techniques involving processive consensus. In some embodiments, reporter elements in certain units may be detected 2, 3, 4, 5, 6, 7, 8, 9, 10, or more times. The type of reporter element at a unit may be determined to be the type that is most frequently detected.

As such, in an example, at block 1650, the following actions may be taken: Detect, in the nanopore, the types of reporter elements in the second subset.

In embodiments, process 1600 may further include passing the compound entirely out of the nanopore. The compound may pass out of the nanopore during a number of voltage pulses with the same polarity as the voltage pulses of the first number of voltage pulses. For instance, the compound may pass out of the nanopore during a bright period. The compound may pass entirely out of the nanopore during the applying of the third number of voltage pulses.

Additional numbers of voltage pulses of alternating polarities may be applied. For example, a fourth number of voltage pulses may displace the compound in the second direction. A fifth number of voltage pulses may then displace the compound in the first direction. These cycles of voltage pulses may continue until the compound passes out of the nanopore.

In some embodiments, a clearing voltage at a fourth level may be applied across the nanopore to pass the compound entirely out of the nanopore. The fourth level may be greater than the first level, the second level, and the third level. In some embodiments, the clearing voltage may be applied for a longer duration than the duration of the first number, second number, and third number of pulses.

In some embodiments, several nanopores (e.g., in an array) may be used to sequence several nucleic acid molecules. The compound may be a first compound of a plurality of compounds. The plurality of compounds may be created from a plurality of target nucleic acid molecules. The nanopore may be a first nanopore of a plurality of nanopores. Each compound of the plurality of compounds may be in one nanopore of the plurality of nanopores. The first number of voltage pulses at the first level, the second number of voltage pulses at the second level, and the third number of voltage pulses at the third level may be applied to the plurality of nanopores. The pulses may be applied in the same order as for the first compound. Reporter elements in each compound of the plurality of compounds may be detected. A plurality of sequences of the plurality of nucleic acid molecules may be determined. The size distribution of the plurality of sequences may have a mode greater than 300 nt. The mode may be 200 to 300 nt, 300 to 400 nt, 400 to 500 nt, or greater than 500 nt.

In some embodiments, clearing voltages may be applied to certain nanopores and not other nanopores, similar to the process described in section [symbol] . The first number of voltage pulses, the second number of voltage pulses at the second level, and the third number of voltage pulses at the third level to the plurality of nanopores may be applied to the plurality of nanopores. A first portion of the plurality of compounds may be determined to be displaced in a first portion of the plurality of nanopores by the first number of voltage pulses, the second number of voltage pulses, or the third number of voltage pulses. The first portion of the plurality of compounds may not be stuck in the nanopore. A clearing voltage at a fourth level may be applied across each nanopore of a second portion of the plurality of nanopores. The applied clearing voltage may pass a second portion of the plurality of compounds entirely out of the respective nanopore of the plurality of nanopores. The fourth level may be greater than the first level, the second level, and the third level. In some embodiments, the clearing voltage may be applied for a longer duration the duration of any of the voltage pulses. The second portion of the plurality of nanopores may not include nanopores in the first portion of the plurality of nanopores.

In some embodiments, the first portion of the plurality of compounds may be determined to not be displaced in a first portion of the plurality of nanopores by the first number of voltage pulses, the second number of voltage pulses, or the third number of voltage pulses. The first portion of the plurality of compounds may be stuck in the nanopore. A clearing voltage at a fourth level may be applied across each nanopore of the first portion of the plurality of nanopores.

Compounds may be determined to be stuck when there is no characteristic change in the measured electrical signal moving from one reporter element to the next. When moving from one reporter element to the next, there is a characteristic change in the measured electrical signal. When stuck at a particular reporter element, there is no change in the electrical signal expected after applying the translocation voltage pulse. A high enough sampling rate would be used to distinguish between stuck compounds and compounds that are moving but have consecutive types of reporter elements.

Process 1600 may include additional implementations, such as any single implementation or any combination of implementations described herein and/or in connection with one or more other processes described elsewhere herein.

Although FIG. 16 shows example blocks of process 1600, in some implementations, process 1600 may include additional blocks, fewer blocks, different blocks, or differently arranged blocks than those depicted in FIG. 16. Additionally, or alternatively, two or more of the blocks of process 1600 may be performed in parallel.

### IV. Example Systems

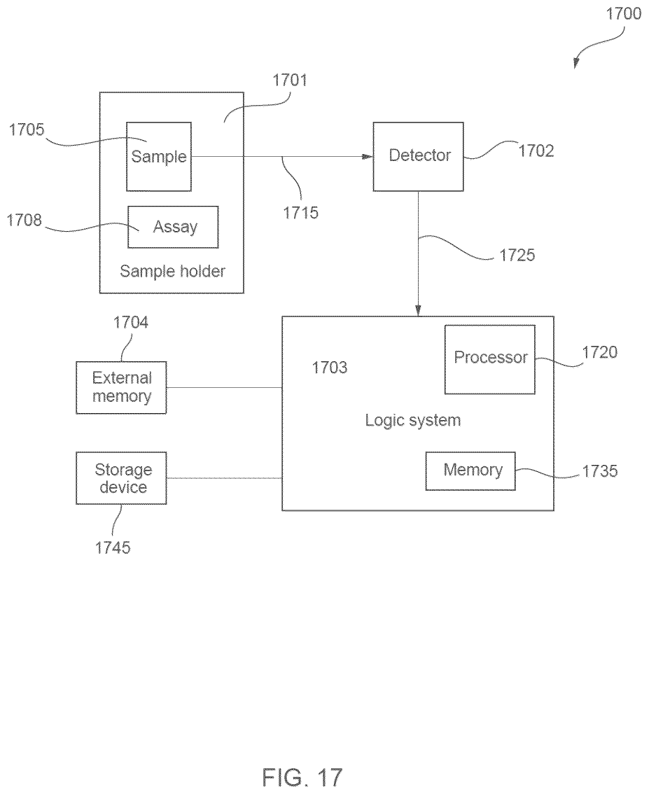

FIG. 17 illustrates a measurement system 1700 according to an embodiment of the present invention. The system as shown includes a sample 1705, such as Xpandomers within a sample holder 1701, where sample 1705 can be contacted with an assay 1708 to provide a signal of a physical characteristic 1715. Assay 1708 may include sequencing by expansion with a nanopore. An example of a sample holder can be a well plate that includes Xpandomers. Physical characteristic 1715 (e.g., a voltage, a current, or other electrical characteristic), from the sample is detected by detector 1702. Detector 1702 can take a measurement at intervals (e.g., periodic intervals) to obtain data points that make up a data signal. In one embodiment, an analog-to-digital converter converts an analog signal from the detector into digital form at a plurality of times. Detector 1702 may be a voltage or current measurement device. Sample holder 1701 and detector 1702 can form an assay device. A data signal 1725 is sent from detector 1702 to logic system 1703. Data signal 1725 may be stored in a local memory 1735, an external memory 1704, or a storage device 1745.

Logic system 1703 may be, or may include, a computer system, ASIC, microprocessor, etc. It may also include or be coupled with a display (e.g., monitor, LED display, etc.) and a user input device (e.g., mouse, keyboard, buttons, etc.). Logic system 1703 and the other components may be part of a stand-alone or network connected computer system, or they may be directly attached to or incorporated in a device (e.g., a sequencing device) that includes detector 1702 and/or sample holder 1701. Logic system 1703 may also include software that executes in a processor 1720. Logic system 1703 may include a computer readable medium storing instructions for controlling system 1700 to perform any of the methods described herein. For example, logic system 1703 can provide commands to a system that includes sample holder 1701 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

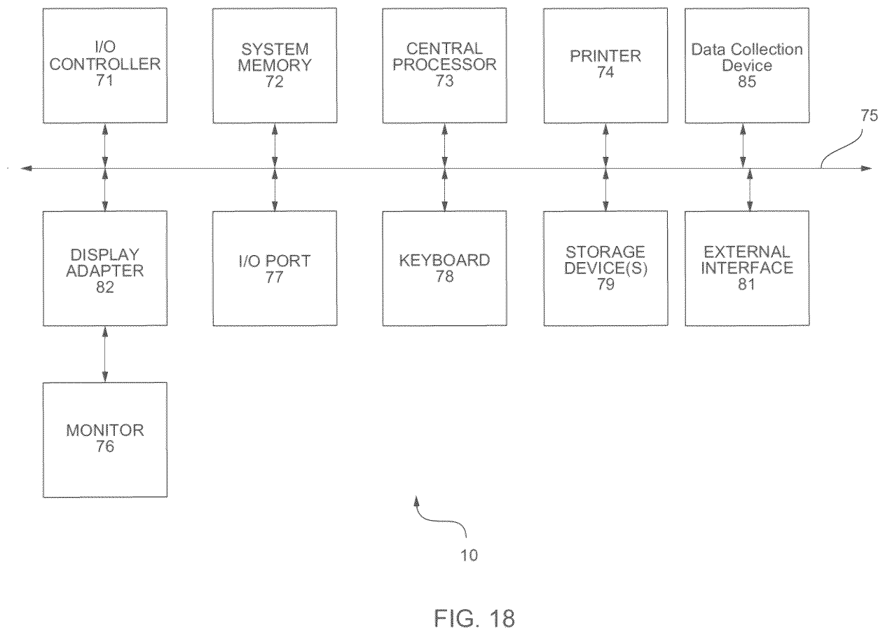

Any of the computer systems mentioned herein may utilize any suitable number of subsystems. Examples of such subsystems are shown in FIG. 18 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones, other mobile devices, and cloud-based systems.

The subsystems shown in FIG. 18 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, Thunderbolt, Lightning). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g., an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an”, or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art.

1. A method for sequencing a target nucleic acid molecule, the method comprising:
applying a first number of voltage pulses at a first level across a nanopore to displace a compound a first distance in a first direction through the nanopore, the compound created from the target nucleic acid molecule, wherein:
the compound comprises a plurality of units,
each unit of the plurality of units comprises one type of reporter element of a plurality of types of reporter elements,
each type of reporter element corresponds to an identity of a nucleotide in the target nucleic acid molecule, and
applying the first number of voltage pulses passes a first subset of the plurality of units through the nanopore;
detecting, in the nanopore, the types of reporter elements in the first subset;
applying a second number of voltage pulses at a second level across the nanopore to displace the compound a second distance in a second direction through the nanopore, wherein:
the first direction is opposite the second direction,
the voltage pulses of the first number of voltage pulses have an opposite polarity as the voltage pulses of the second number of voltage pulses,
the second distance is less than the first distance, and
the second number is less than the first number;
applying a third number of voltage pulses at a third level across the nanopore to displace the compound a third distance in the first direction through the nanopore, wherein:
applying the third number of voltage pulses passes a second subset of the plurality of units through the nanopore,
the second subset and the first subset comprise some of the same units,
the second subset comprises units not in the first subset, and
the third distance is greater than the second distance; and
detecting, in the nanopore, the types of reporter elements in the second subset.

2. The method of claim 1, further comprising:
applying a clearing voltage at a fourth level across the nanopore to pass the compound entirely out of the nanopore, wherein the fourth level is greater than the first level, the second level, and the third level.

3. The method of claim 1, wherein:
the compound is a first compound of a plurality of compounds,
the plurality of compounds is created from a plurality of target nucleic acid molecules,
the nanopore is a first nanopore of a plurality of nanopores, and
each compound of the plurality of compounds is in one nanopore of the plurality of nanopores,
the method further comprising:
applying the first number of voltage pulses at the first level, the second number of voltage pulses at the second level, and the third number of voltage pulses at the third level to the plurality of nanopores.

4. The method of claim 3, further comprising:
determining a plurality of sequences of the plurality of target nucleic acid molecules.

5. The method of claim 4, wherein:
the size distribution of the plurality of sequences has a mode greater than 300 nt.

6. The method of claim 3, further comprising:
applying the first number of voltage pulses at the first level, the second number of voltage pulses at the second level, and the third number of voltage pulses at the third level to the plurality of nanopores;
determining that a first portion of the plurality of compounds is being displaced in a first portion of the plurality of nanopores by the first number of voltage pulses, the second number of voltage pulses, or the third number of voltage pulses,
applying a clearing voltage at a fourth level across each nanopore of a second portion of the plurality of nanopores to pass a second portion of the plurality of compounds entirely out of the respective nanopore of the plurality of nanopores, wherein:
the fourth level is greater than the first level, the second level, and the third level, and
the second portion of the plurality of nanopores does not comprise nanopores in the first portion of the plurality of nanopores.

7. The method of claim 1, further comprising determining a sequence of the target nucleic acid molecule.

8. The method of claim 7, wherein determining the sequence of the target nucleic acid molecule comprises:
for one or more units in both the first subset and the second subset, detecting the same type of reporter element.

9. The method of claim 1, further comprising passing the compound entirely out of the nanopore.

10. The method of claim 9, wherein passing the compound entirely out of the nanopore occurs during the applying of the third number of voltage pulses.

11. The method of claim 1, wherein:
each unit of the plurality of units comprises a translocation control element,
applying the first number of voltage pulses passes a first number of translocation control elements through the nanopore, and
the first number of voltage pulses equals the first number of translocation control elements.

12. The method of claim 11, further comprising:
applying a voltage at a fourth level across the nanopore to displace the compound a fourth distance in the first direction through the nanopore in between voltage pulses of the first number of voltage pulses, wherein:
the voltage at the fourth level is the same polarity as the voltage pulses of the first number of voltage pulses,
the fourth level is less than the first level,
the compound after being displaced the fourth distance has a translocation control element in the nanopore.

13. The method of claim 1, wherein the second level is greater than the first level.

14. The method of claim 1, further comprising:
measuring signal values for a nanopore having a voltage applied across the nanopore when reporter elements in the first subset of the plurality of units are in the nanopore;
determining, using the signal values, the types of reporter elements in the first subset, thereby determining the identities of nucleotides in the target nucleic acid molecule.

15. The method of claim 1, wherein the first subset of the plurality of units comprises 30 or more units.

16. The method of claim 1, wherein the third level is equal to the first level.

17. The method of claim 1, wherein the target nucleic acid molecule is longer than 200 nt.

18. The method of claim 1, wherein the first number of voltage pulses is 30 or more.

19. The method of any one of claim 1, wherein the first number of voltage pulses may exceed the second number of voltage pulses by 5 or more.

20. A computer product comprising a non-transitory computer readable medium storing a plurality of instructions that when executed control a computer system to perform the method of claim 1.

21. A system comprising:
the computer product of claim 20, and
one or more processors for executing instructions stored on the computer readable medium.

22. A system comprising means for performing the method of claim 1.

23. A system comprising one or more processors configured to perform the method of claim 1.

24. A system comprising modules that respectively perform the steps of the method of claim 1.
